(19) United States
(12) Patent Application Publication
(10) Pub. No.: US 2025/0084230 A1
(43) Pub. Date: Mar. 13, 2025

(54) METHOD FOR RECOVERING MONOMER

(71) Applicant: HONDA MOTOR CO., LTD., Tokyo (JP)

(72) Inventors: Ryosuke KODAIRA, Saitama (JP); Yoshitaka SUZUKI, Saitama (JP); Ryo NAKAI, Saitama (JP)

(21) Appl. No.: 18/672,014

(22) Filed: May 23, 2024

(30) Foreign Application Priority Data
Sep. 13, 2023 (JP) 2023-148772

(51) Int. Cl. C08J 11/16; C08J 11/08; C08J 11/24; C08J 11/26

(52) U.S. Cl. CPC C08J 11/16; C08J 11/08; C08J 11/24; C08J 11/26; C08J 2367/02; C08J 2375/04

(57) ABSTRACT

Provided is a method for recovering a monomer from a resin composition comprising a polyester and a polyurethane, the method comprising: depolymerizing the polyester and the polyurethane contained in the resin composition in the presence of a solvent capable of swelling the polyester and the polyurethane, an alcohol, and a base; extracting a depolymerized product of polyester and a depolymerized product of polyurethane with a hydrophilic solvent and a hydrophobic solvent, respectively; and acid precipitating the depolymerized product of the polyester extracted with the hydrophilic solvent.

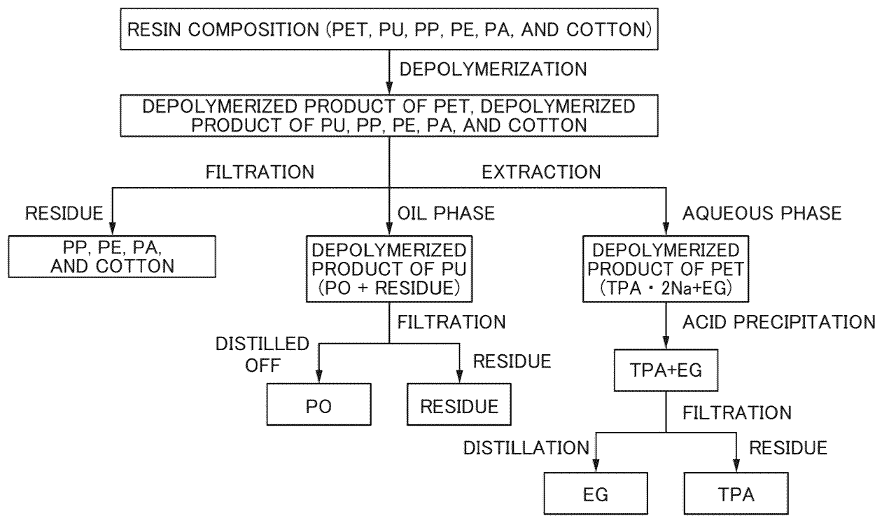

This application is based on and claims the benefit of priority from Japanese Patent Application No. 2023-148772, filed on 13 Sep. 2023, the content of which is incorporated herein by reference.

### BACKGROUND OF THE INVENTION

### Field of the Invention

The present invention relates to a method for recovering a monomer from a resin composition comprising a polyester and a polyurethane.

### Related Art

In recent years, efforts have been made to reduce the generation of waste through prevention, reduction, recycling, and reuse of waste. To achieve this, research and development have been conducted on a method for recovering a monomer from waste containing a polyester.

Patent Document 1 describes a method for separating and refining dimethyl terephthalate and ethylene glycol, the method comprising collecting, from the market, a commercially available product containing a polyester as a main component and/or waste generated in the process of producing the product and containing the polyester as a main component; and carrying out a depolymerization reaction with ethylene glycol and a transesterification reaction with methanol, using the above-mentioned product and waste as raw materials.
Patent Document 1: Japanese Unexamined Patent Application, Publication No. 2002-167469

### SUMMARY OF THE INVENTION

However, in the process described in Patent Document 1, in a case of monomer recovery from a resin composition containing a polyester and a polyurethane, a recovery rate and a recovery concentration of dicarboxylic acid derived from the polyester are insufficient.

An object of the present invention is to provide a method for recovering a monomer from a resin composition containing a polyester and a polyurethane, the method being capable of improving the recovery rate and the recovery concentration of dicarboxylic acid derived from the polyester.

A first aspect of the present disclosure relates to a method for recovering a monomer from a resin composition including a polyester and a polyurethane, the method including: depolymerizing the polyester and the polyurethane contained in the resin composition in the presence of a solvent capable of swelling the polyester and the polyurethane, an alcohol, and a base; extracting a depolymerized product of the polyester and a depolymerized product of the polyurethane with a hydrophilic solvent and a hydrophobic solvent, respectively; and acid precipitating the depolymerized product of the polyester extracted with the hydrophilic solvent.

A second aspect of the present disclosure relates to the method for recovering a monomer as described in the first aspect, in which the alcohol is methanol, the solvent capable of swelling the polyester and the polyurethane is tetrahydrofuran, and the base is sodium hydroxide.

A third aspect of the present disclosure relates to the method for recovering a monomer as described in the first or second aspect, in which the hydrophilic solvent is water and the hydrophobic solvent is ethyl acetate.

A fourth aspect of the present disclosure relates to the method for recovering a monomer as described in any one of the first to third aspects, in which the temperature at which the polyester and the polyurethane are depolymerized is from 45° C. to 75° C.

According to the present invention, it is possible to provide a method of recovering a monomer from a resin composition including a polyester and a polyurethane, the method capable of improving a recovery rate and a recovery concentration of a dicarboxylic acid derived from the polyester.

### BRIEF DESCRIPTION OF THE DRAWINGS

FIG. is a diagram showing a method for recovering a monomer of Example 1.

### DETAILED DESCRIPTION OF THE INVENTION

Hereinafter, embodiments of the present invention will be described.

The monomer recovery method according to one embodiment of the present disclosure relates to a method of recovering a monomer from a resin composition containing a polyester and a polyurethane.

Examples of the polyester include polyethylene terephthalate and polybutylene terephthalate. The polyurethane includes, for example, a polyether polyol residue. In addition to the polyester and the polyurethane, the resin composition may further include a resin such as a polyolefin (polypropylene, polyethylene, or the like), a polyamide (polyamide 6, polyamide 66, or the like), cotton, or the like and may further include an additive such as a filler.

The resin composition is not particularly limited, and examples thereof include shredder dust obtained by crushing a waste vehicle. The shredder dust includes, for example, a polyester fiber and a foamed polyurethane.

In the monomer recovery method of the present embodiment, first, the polyester and the polyurethane contained in the resin composition are depolymerized in the presence of a solvent capable of swelling the polyester and the polyurethane, an alcohol, and a base.

Examples of the solvent capable of swelling the polyester and the polyurethane include, but are not particularly limited to, methyl ethyl ketone, tetrahydrofuran, and dichloromethane. Among these, tetrahydrofuran is preferable from the viewpoint of reactivity of the depolymerization reaction.

The alcohol is not particularly limited as long as it is capable of transesterifying polyester and alcohol, and examples thereof include methanol and ethanol. Among these, methanol is preferable from the viewpoint of reactivity of the transesterification reaction.

The base is not particularly limited as long as it can depolymerize a polyester and a polyurethane, and examples thereof include sodium hydroxide, potassium hydroxide, and potassium t-butoxide. Among these, sodium hydroxide is preferable from the viewpoint of reactivity of the depolymerization reaction.

Depolymerization temperature is preferably from 45° C. to 75° C. and more preferably from 45° C. to 65° C. When the depolymerization temperature is 45° C. or higher, depolymerization time can be shortened, and when the temperature is 75° C. or lower, a side reaction such as depolymerization of polyamide and depolymerization of cotton can be suppressed. The depolymerization time is not particularly limited, but is, for example, 5 hours or more and 20 hours or less.

Next, the depolymerized product of polyester and the depolymerized product of polyurethane are extracted with a hydrophilic solvent and a hydrophobic solvent, respectively.

Examples of the depolymerized product of polyester include a dicarboxylate salt (e.g., disodium terephthalate) and a dialcohol (e.g., ethylene glycol and butylene glycol). Examples of the depolymerized product of polyurethane include a polyether polyol.

The hydrophilic solvent is not particularly limited as long as it can extract a depolymerized product of polyester, and examples thereof include water. The hydrophobic solvent is not particularly limited as long as it can extract a depolymerized product of polyurethane and examples thereof include ethyl acetate, propyl acetate, butyl acetate, methyl ethyl ketone, and cyclohexane. Among these, ethyl acetate is preferable from the viewpoint of extractability of the depolymerized product of polyurethane.

When the resin composition further contains a resin other than a polyester and a polyurethane or an additive, it is preferable to collect residue by filtration after extraction of the depolymerized product of polyester and the depolymerized product of polyurethane. When it is necessary to further sort the collected residue, the residue is subjected, for example, to specific gravity sorting.

Next, the depolymerized product of polyester extracted with the hydrophilic solvent is subjected to acid precipitation. With this operation, dicarboxylic acid is precipitated. The dicarboxylic acid is recovered, for example, by drying the filtrate after filtration. Therefore, the recovery rate and the recovery concentration of the dicarboxylic acid are improved. At this time, the dialcohol is recovered, for example, by distilling the filtrate. On the other hand, the depolymerized product of polyurethane extracted with hydrophobic solvent is recovered by distilling off the hydrophobic solvent from the filtrate.

The pH during the acid precipitation is preferably 4 or less and more preferably 3 or less. When the pH during the acid precipitation is 4 or less, the dicarboxylic acid is sufficiently precipitated. The acid used for the acid precipitation is not particularly limited, and examples thereof include hydrochloric acid, sulfuric acid, and nitric acid. Among these, sulfuric acid is preferable from the viewpoint of precipitation of dicarboxylic acid.

Although the embodiments of the present invention have been described above, the present invention is not limited to the above-described embodiments, and the above-described embodiments may be appropriately modified within the scope of the gist of the present invention.

### EXAMPLES

Hereinafter, Examples of the present invention will be described, but the present invention is not limited to the Examples.

### [Resin Composition]

4 g of each of polyethylene terephthalate (PET), polyurethane (PU) containing a polyether polyol (PO) residue, polypropylene (PP), polyethylene (PE), polyamide 6 (PA6), and cotton were mixed to obtain a resin composition.

### Example 1

As shown in FIG., monomers derived from polyester were recovered from the resin composition. First, 50 mL of tetrahydrofuran, 50 mL of methanol, and 2 g of sodium hydroxide were added to the resin composition, and the obtained mixture was stirred at 65° C. for 20 hours to depolymerize PET and PU. Next, the solvent was distilled off using an evaporator, then 200 ml of water was added and a depolymerized product of PET was extracted. Next, an operation of extracting the depolymerized product of PU into 150 mL of ethyl acetate using a separatory funnel was repeated three times to collect an aqueous phase, followed by filtration under reduced pressure to recover an oil phase. Next, the obtained residue was washed with a mixed solvent (20 mL of water/20 mL of ethyl acetate) and was dried overnight at 80° C. to recover a mixture of PP, PE, PA6, and cotton. After a mixed solvent (20 ml of water/20 mL of ethyl acetate) was added to the filtrate, an aqueous phase and an oil phase were collected using a separatory funnel. Here, the aqueous phase included disodium terephthalate (TPA·2Na) and ethylene glycol (EG) and the oil phase included PO.

After 100 mL of saturated brine was added to the oil phase, the oil phase was washed and recovered using a separatory funnel. Next, magnesium sulfate and 20 mL of ethyl acetate were added to dry the oil phase, followed by filtration under reduced pressure, and then the magnesium sulfate was washed with 20 mL of ethyl acetate. Next, 20 mL of ethyl acetate was added to the filtrate, the solvent was distilled off using an evaporator, and the residue was dried overnight at 80° C. to recover PO.

On the other hand, a 2 N sulfuric acid aqueous solution was added to the aqueous phase until the pH reached 2 to precipitate terephthalic acid (TPA), followed by filtration under reduced pressure. Next, the residue was washed with 20 mL of methanol and dried overnight at 80° C. to recover TPA. As a result, the recovery rate of TPA was 99 mass % and the recovery concentration of TPA was 98 mass %. The filtrate was distilled using an evaporator and ethylene glycol (EG) was recovered.

What is claimed is:

1. A method for recovering a monomer from a resin composition comprising a polyester and a polyurethane, the method comprising:
depolymerizing the polyester and the polyurethane contained in the resin composition in a presence of a solvent capable of swelling the polyester and the polyurethane, an alcohol, and a base;
extracting a depolymerized product of the polyester and a depolymerized product of the polyurethane with a hydrophilic solvent and a hydrophobic solvent, respectively; and
acid precipitating the depolymerized product of the polyester extracted with the hydrophilic solvent.

2. The method for recovering a monomer according to claim 1,
wherein the alcohol is methanol,
the solvent capable of swelling the polyester and the polyurethane is tetrahydrofuran, and
the base is sodium hydroxide.

3. The method for recovering a monomer according to claim 1,
wherein the hydrophilic solvent is water and
the hydrophobic solvent is ethyl acetate.

4. The method for recovering a monomer according to claim 1,
wherein the temperature at which the polyester and the polyurethane are depolymerized is from 45° C. to 75° C.
